(19) United States
(12) Patent Application Publication
(10) Pub. No.: US 2025/0354918 A1
(43) Pub. Date: Nov. 20, 2025

(54) GAS DETECTION DEVICE COMPRISING MULTIPLE DETECTORS FOR DIFFERENT TARGET GASES

(71) Applicant: Drãger Safety AG & Co. KGaA, Lübeck (DE)

(72) Inventor: Sebastian Draack, Lübeck (DE)

(21) Appl. No.: 19/206,242

(22) Filed: May 13, 2025

(30) Foreign Application Priority Data
May 17, 2024 (DE) 102024113935.8

(51) Int. Cl. G01N 21/31; G01N 33/00; G08B 21/12

(52) U.S. Cl. CPC G01N 21/31; G01N 33/0006; G01N 33/0063; G08B 21/12

(57) ABSTRACT

The present disclosure relates to a gas detection device and a gas detection method which are capable of detecting N target gases in a gas sample. The gas sample is fed into a measuring chamber. M detectors each generate a signal which correlates to the concentration of at least one of the N target gases to be detected in the gas sample. A determiner comprises M inputs and N outputs. A signal, which depends on the signal of the associated measuring detector, is applied at each input of the determiner. Each output supplies information on the concentrations of the associated target gas. The determiner is trained by applying a learning method to a sample with a plurality of sampling elements. Each sample element contains M values for M signals from measuring detectors and N values of N target gas concentrations.

### CROSS-REFERENCE TO RELATED APPLICATIONS

This application claims the priority of German Patent Application No. 102024113935.8, filed on May 17, 2024, and titled “GAS DETECTION DEVICE COMPRISING MULTIPLE DETECTORS FOR DIFFERENT TARGET GASES,” which is hereby incorporated by reference in its entirety for all nonlimiting purposes.

### SUMMARY

The present disclosure relates to a gas detection device which comprises a plurality of measuring detectors and is capable of detecting various target gases in a gas sample. Furthermore, the present disclosure comprises a gas detection method which is carried out using such a gas detection device.

A typical application of a gas detection device is the following: At least one target gas, optionally simultaneously a plurality of target gases, can occur in a spatial region. In one application, the or every target gas is dangerous (harmful) for a person, for example because the target gas is flammable or toxic. At least one target gas can also be a gas that is vital for life, in particular oxygen, or an anesthetic. The gas detection device is capable of detecting the target gas or each target gas of a given target gas set and optionally determining the relevant concentration of each target gas.

The present disclosure is based on the object of providing a gas detection device and a gas detection method which are capable of simultaneously detecting a plurality of predetermined target gases in a gas sample and which are in many cases more reliable than known gas detection devices and gas detection methods.

The object is achieved by a gas detection device and a gas detection method having the features described herein. Advantageous embodiments are specified in the claims. Advantageous embodiments of the gas detection device according to the present disclosure are, where applicable, also advantageous embodiments of the gas detection method according to the present disclosure, and vice versa.

A target gas set with N different target gases is specified. It is therefore specified which N different target gases may occur and are to be detected. The task of detecting a target gas can result in that the presence of the target gas is currently ruled out. N is a predetermined number being greater than or equal to 2. The gas detection device according to the present disclosure and the gas detection method according to the present disclosure are capable of detecting each one of these N target gases in a gas sample and preferably additionally of determining the respective concentration of each target gas to be detected.

The gas detection device according to the present disclosure comprises a measuring chamber. This measuring chamber is able to hold (keep) a gas sample to be examined. This gas sample may be free of any target gas or may contain at least one of the N target gases. Preferably, the gas sample comes from a spatial region to be monitored.

Furthermore, the gas detection device comprises a detection arrangement. The detection arrangement comprises M measuring detectors, where M is a predetermined number being greater than or equal to 2 and preferably greater than or equal to 3. The number M can be greater than, equal to or less than N and is preferably at least as large as N. Each measuring detector of the detection arrangement is capable of generating a respective signal. This signal correlates with (depends on) the concentration of at least one of the N target gases in a gas sample, wherein the gas sample is contained in the measuring chamber. Therefore, the signal is influenced by the concentration of at least one target gas, preferably by several target gas concentrations. Conversely, the gas detection device is configured as follows: Each target gas to be detected influences the signal of at least one measuring detector of the detection arrangement. The signal of this measuring detector depends on the concentration of this target gas in the gas sample. As a rule, this signal does not depend on the concentration of the or at least one other target gas in the gas sample and is therefore not influenced by this other target gas.

The detection arrangement is configured to achieve the following:
If the gas sample is free of any target gas to be detected, each measuring detector generates a respective reference signal. “Free of a target gas” means: The concentration of the target gas is below a construction-related detection limit.
However, if the gas sample has a different chemical composition, at least one measuring detector generates a deviating signal, namely a signal that deviates from the reference signal of this measuring detector. “Different chemical composition” means: At least one of the N target gases occurs in the gas sample at a concentration above a detection limit. The gas sample then contains at least one target gas to be detected. The respective signal generated by a measuring detector is called a first signal. The first signal can equal the reference signal or can deviate from the reference signal.

The gas detection device also comprises a signal-processing determiner (determination device). As a rule, the determiner is implemented as software and can run on a processor. It can also be a signal-processing unit. For each measuring detector of the detection arrangement, the determiner comprises one respective input, thus a total of M different inputs (M is the number of detectors). Each of these M inputs is therefore associated with a measuring detector of the detection arrangement. Optionally, the determiner comprises at least one additional input. Furthermore, the determiner comprises one output for each target gas to be detected, i.e., a total of N different outputs for the N target gases of the specified target gas set.

The gas detection method is carried out using such a gas detection device.

The gas detection device is configured as follows, and the gas detection method comprises the following steps: The first signal, which has been or is generated by a measuring detector, is applied to that input of the determiner that is associated with this measuring detector. More generally: A signal is applied to this input wherein the applied signal depends on the first signal of the associated measuring detector. The signal of a reference detector or the signal of a sensor for an environmental condition is present at a respective optional additional input.

That output of the determiner that is associated with a target gas of the target gas set provides a value that depends on the concentration of this target gas in the gas sample. The value is, for example, the target gas concentration itself or a suitably normalized or standardized target gas concentration or an indication of whether the target gas concentration is above a specified lower concentration limit or not, i.e. is present or not. In one embodiment, the first signal of a measurement detector is normalized or standardized using the signal of an optional reference detector, this embodiment being described below.

Therefore, the determiner receives the M first signals of the M measuring detectors via its M inputs and optionally via at least one further input another signal, e.g., of the optional reference detector and/or of a sensor for an environmental condition. The determiner provides at its N outputs the N values for the N target gas concentrations, wherein the N target gases to be detected have these concentrations in the gas sample. Of course, the gas sample can be free of at least one target gas to be detected or even free of each of the N target gases.

The determiner is created as follows: A machine learning method is applied to a training sample. This application automatically trains the determiner. The training sample used for training comprises a plurality of sample elements. Each sample element results from a respective gas sample. The respective chemical composition of each gas sample and the M signals of the M measuring detectors for this chemical composition are known, e.g. from a measurement performed in advance. Each sample element comprises, on the one hand, M values, namely one respective value for the M measuring detectors, and, on the other hand, N values, namely one respective value for the N target gas concentrations. The value for a measuring detector depends on the first signal that this measuring detector generates. The value for a target gas depends on the concentration of this target gas in that gas sample which yielded the sample element. Each sample element refers to a specific combination of N target gas concentrations. Each measuring detector has generated this signal if the gas sample has this combination of N target gas concentrations.

The training sample was generated in advance. During the generation, a respective gas sample having a known chemical composition was used for each sample element. “Known chemical composition” means the following: The relevant concentration of each target gas in each gas sample is known. The M signals of the M measuring detectors are generated with the same gas detection device that is later used to analyze an unknown gas sample or with a gas detection device having the same design and implementation.

The determiner is created in advance in a training phase. It is possible to retrain the determiner after a use of the gas detection device. In particular, through the retraining the determiner is adapted to a changed state of the gas detection device or to a changed operating or ambient condition.

The gas detection method according to the present disclosure comprises the corresponding steps.

As a rule, for a particular application or use it is known which target gases can occur in a spatial region to be monitored and therefore in a gas sample from this spatial region. Therefore, the N target gases to be detected are usually known beforehand and are therefore predetermined. Thanks to the present disclosure, it is not necessary to use at least two different gas detection devices to detect these N target gases. Rather, the gas detection device according to the present disclosure is able to examine the gas sample in the measuring chamber simultaneously for N different target gases and to supply for each target gas information on the respective target gas concentration. The gas detection device is at least able to automatically decide for each of the N target gases whether or not the gas sample in the measuring chamber contains this target gas at a concentration above a predetermined lower concentration limit, in particular a detection limit.

The gas detection device according to the present disclosure comprises M measuring detectors. The determiner comprises one respective input for each measuring detector and respective one output for each target gas to be detected. In particular thanks to this feature, it is possible for all measuring detectors to use the same measuring principle. This advantage facilitates a manufacture and maintenance of the gas detection device compared to a gas detection device that comprises at least two differently operating measuring detectors.

In addition, in many cases it is possible to use at least one component of the gas detection device for all M measuring detectors. As a rule, it is sufficient for one and the same measuring chamber to collect a gas sample and this gas sample is analyzed for the N target gases. In the case of photoelectric (optoelectric) or photoacoustic measuring detectors, it is possible to use a single radiation source or sound source for all measuring detectors. This would not be possible if for each target gas a measuring detector had to be used that is tailored to this target gas and, for example, applies a measuring principle that is particularly suitable for this target gas. The gas detection device according to the present disclosure requires-if at all-only one display unit, one input unit and only one single connection to a stationary power supply network or only one own power supply unit.

Thanks to the present disclosure, it is not necessary to switch the gas detection device between different modes during operation, wherein each mode is associated with a target gas and wherein the gas detection device, while operating in the mode associated with a target gas, is configured to detect this target gas and optionally to measure the concentration of this target gas. If a user were to set these modes, this setting would require user action. If the user forgets a mode or a mode cannot be set due to a defect, there is a risk that a harmful target gas or the absence of a target gas that is vital for life will not be detected. If the gas detection device were to automatically switch from the one mode to another, this would require a corresponding control unit, which may fail or be faulty. In both embodiments, the procedure with the different modes requires more time to analyze the gas sample for each target gas to be detected than the gas detection device according to the present disclosure requires. Thus, the gas detection device according to the present disclosure has a shorter reaction time (response time). In addition, readjustment would sometimes be necessary.

According to the present disclosure, the determiner is generated using a training sample. In many cases, it is therefore not necessary to determine analytically beforehand how a target gas affects the detection arrangement. Rather, the training sample is generated using the same gas detection device or at least a gas detection device of the same design and/or implementation as the gas detection device that is later used to analyze the gas sample in the measuring chamber. In addition, in many cases, a result of the gas detection device according to the present disclosure depends relatively little on cross-sensitivities between different target gases.

As already explained, the determiner is trained using a training sample. The training sample comprises a plurality of sample elements. Each sample element is generated using a gas sample having a known chemical composition. The sample element comprises the N known target gas concentrations and further comprises the respective signal of each measuring detector. In one embodiment, the respective gas sample used for generating a plurality of sample elements, preferably for each sample element, has only one target gas to be detected having a concentration above a lower concentration limit (e.g., detection limit). The relevant signal provided by a measuring detector for this sample element is divided by the concentration of the target gas contained in the gas sample for this sample element. The sample element comprises M values for the M first signals of the M measuring detectors and additionally a vector of N values, namely one respective value for the concentration of the N target gas. In the embodiment just described, this vector consists of a 1 and N−1 zeros. Optionally, the respective first signal of a measuring detector is additionally divided by a signal of the optional reference detector or otherwise normalized using the reference detector signal. The reference detector has provided this signal for this gas sample.

Different embodiments are possible as to how the determiner is generated using the training sample. In one embodiment, the determiner comprises a neural network. This neural network is generated using the training sample. Methods known in the art for training a neural network can be applied to the present disclosure.

In a preferred embodiment, however, the fact is exploited that in many cases the particular influence (impact) of each target gas on the signals of the measuring detectors does not depend in a relevant manner on the influence of another target gas (relatively low cross sensitivity). With sufficient accuracy, it can often be assumed that the absorption spectra of the target gases are superimposed linearly. A certain combination of signal values of the M measuring detectors is therefore often only determined (caused, effected) by a mixture of the N target gases with a certain mixing ratio. The mixing ratio determines the signal value combination. Of course, this also applies if at least one of the N target gases is not contained in the mixture at all. The signal value combination is thus a “fingerprint” of this gas mixture with this mixing ratio. In many cases, it also applies with sufficient accuracy that the influence of the target gases on a measuring detector can be described as a weighted combination of the target gas concentrations in a gas sample, wherein the weighting factors of this weighted combination depend on the absorption behavior of the target gas and on the design and implementation of the measuring detectors and are therefore constant in use.

In accordance with this embodiment, using the training sample and during the training of the determiner, a trained matrix A is generated such that the product A x deviates for the training sample as little as possible from a vector b, ideally matches b. Here, A is a matrix comprising M rows and N columns, namely one row for the M measuring detectors and one column for the N target gases to be detected. The vector x comprises N vector elements, which depend on the respective concentrations of the N target gases. The vector b comprises M vector elements, which depend on the M first signals of the M measuring detectors. Each sample element of the training sample yields a respective vector x and one respective vector b. Preferably, each element of the vector b depends on exactly one respective measuring detector. The vectors x and b are obtained in advance using the training sample, and the matrix A is generated on the basis of the vectors x and b.

Preferably the trained matrix A is generated such that for the training sample an indicator for a deviation between the product A x and the vector b is minimized. Preferably, the indicator for the deviation yields an error function, and a minimization procedure is applied on this error function, in particular an iterative minimization procedure.

The matrix A is generated before the gas detection device is used, and is stored, for example, in a memory of the gas detection device.

While using the gas detection device, a vector b is generated on the basis of the M first signals from the M measuring detectors, and the vector x for the sought concentrations of the N target gases to be detected is generated using the equation x=A⁻¹ b. Here, A⁻¹ is an inverse or pseudoinverse of the matrix A. The used matrix A⁻¹ is generated in advance or during operation by the determinate period

Conventionally, a neural network is a black box. It is often difficult to understand why such a black box delivers a certain result. In particular, the embodiment with the matrix has the following advantages over a neural network:
In contrast to the weighting factors and activation functions of a neural network, the elements of the trained matrix A and the vectors b and x each have a physical unit of measurement and a physical meaning.
The trained matrix A can be checked for plausibility.
In many cases it is possible to deduce how and why the determiner and thus the gas detection device provides a certain result regarding the target gas concentrations. This derivation can be output by means of a suitable output unit in at least one form that is perceptible to a human, at least for an inspection of the device.

For example, for each target gas of the predetermined target gases described herein, the information about the concentration of the associated target gas yielded by the gas detection device is output on an output unit in at least one form which is perceptible by a human. The output unit can be a part of the gas detection device as described herein or can be a part of a remote receiver. Preferably the concentrations of the target gases indicated in the provided information are output simultaneously on the output unit (e.g., all target gas concentrations are output simultaneously).

In one embodiment, for every target gas a respective value range is specified. A target gas concentration outside of this value range may be harmful to a human. The gas detection device generates an alarm if the measured concentration of at least one target gas of the predetermined target gases is outside the value range specified for this target gas. The alarm is output on an alarm unit in at least one form which is perceptible by a human, in particular visibly or acoustically or haptically (e.g., by vibrations). For example, a haptic alarm can often be recognized in a noisy environment and if a user of the gas detection device does not look at the device. The alarm unit can be a part of the gas detection device as described herein or can be arranged remotely. In one embodiment all target gas concentrations are simultaneously output on the output unit wherein a target gas concentration which is outside the respective specified value range is highlighted or otherwise output in another way than the other target gas concentrations.

The first signals of the M measuring detectors are often influenced not only by the chemical composition of a gas sample in the measuring chamber, but also by environmental conditions, in particular by the ambient temperature, the ambient humidity, and/or the ambient pressure, as well as by the current state of a component of the gas detection device, for example the state of a radiation source or an optical filter or its own power supply unit. Therefore, the gas detection device preferably additionally comprises a reference detector. The reference detector can also generate a signal, namely a fourth signal. This fourth signal is influenced by ambient conditions and/or the current state of the gas detection device, but is ideally independent of the chemical composition of the gas sample in the measuring chamber. Ideally, the M measuring detectors react to the current state and the ambient conditions in the same way as the reference detector. The fourth signal from the reference detector can be used to computationally compensate for the influence of the environmental conditions and the influence of the current state, at least approximately compensate. The embodiment with the reference detector eliminates the need to measure an environmental condition directly. However, it is also possible that the gas detection device comprises a sensor for ambient conditions, in particular for the ambient temperature, or is configured to receive and process a signal from a spatially distant sensor for the ambient conditions. In one implementation the determiner comprises an additional input for a measured ambient condition.

According to the present disclosure, M inputs of the determiner are associated with (assigned to) the M measuring detectors of the detection arrangement. In one embodiment, directly the first signal of a specific measuring detector is present at that input that is associated with this measuring detector wherein the present first signal depends on the signal of this measuring detector, e.g. directly the signal of the measuring detector. In one embodiment, these M signals present at the M inputs of the determiner additionally depend on the signal of the reference detector. In a first alternative, the larger the first signal of the associated measuring detector is, the larger is the signal present at the input while the signal of the reference detector remains constant, and the larger is the first signal of the reference detector, the smaller is the signal present at the input while the signal of the measuring detector remains constant. In a second alternative, conversely, the larger is the first signal of the associated measuring detector, the smaller is the applied signal, and the larger the signal of the reference detector is, the larger is the applied signal. For example, the quotient of the first signal of the measuring detector (numerator) and the signal of the reference detector (denominator) is present at the associated input.

In this embodiment, M signals are applied to the determiner and are ideally already computationally compensated for the influence of environmental conditions and the influence of the current state of the gas detection device. This embodiment often leads to better results than if the unadjusted signals from the measuring detectors were present at the inputs.

According to the present disclosure, the determiner comprises M inputs for the M measuring detectors. In one embodiment, the determiner comprises an additional input, namely an input for the reference detector. This embodiment makes it possible to compensate for the influence of environmental conditions and/or the current state that has not yet been eliminated by the embodiment described above, namely in particular has not yet been eliminated by the training.

It is also possible that the gas detection device receives a signal for an environmental condition from a corresponding sensor and the determiner has another input for a signal from this sensor. For example, the neural network is additionally trained with the signal from the reference detector and/or the signal from the sensor for the environmental conditions. Or the trained matrix A has a further row for the reference detector and/or for the signal from the environmental sensor. Optionally, the determiner also comprises an input for a signal of a sensor for an environmental condition.

According to the present disclosure, the gas detection device comprises M measuring detectors and optionally a reference detector. In one embodiment, the M measuring detectors and the optional reference detector are each implemented as a photoelectric (optoelectric) or photoacoustic receiver. The gas detection device comprises a radiation source. This radiation source emits electromagnetic radiation or sound, e.g., ultrasound. In the following, the abbreviated term “electromagnetic radiation” is used, and this also comprises sound and ultrasound. The same radiation source and the same voltage supply for this radiation source are used for all M measuring detectors and for the optional reference detector. Preferably the gas measuring device comprises the measuring chamber and a reference chamber which is free from every target gas.

According to the embodiment with the receivers, the gas detection device is configured as follows: At least a portion of the emitted electromagnetic radiation penetrates at least once the measuring chamber and thus a gas sample in the measuring chamber and hits the measuring detectors. Optionally, the electromagnetic radiation is reflected at least once before it hits (impinges onto) the measuring detectors, so that the optical path is extended. In one embodiment, another part of the electromagnetic radiation is guided past the measuring chamber and thus past a gas sample, preferably through the reference chamber, and hits the reference detector. In a different embodiment, a wavelength filter upstream of the reference detector only allows radiation to pass through in a frequency in which no target gas attenuates radiation. Each detector generates a signal which depends on the intensity of incident electromagnetic radiation.

Each target gas to be detected attenuates electromagnetic radiation that penetrates a gas sample containing this target gas within a target gas frequency band, wherein “attenuation” means a degree of attenuation above a lower attenuation limit. The degree of attenuation depends on the target gas concentration, whereas the target gas frequency band usually depends on the type of target gas, but depends on the target gas concentration only to a relatively small extent or even not at all. This target gas frequency band is often characteristic for the target gas and is known for every target gas that is typically to be detected and thus also for the N specified target gases.

The embodiment just described and also described below takes advantage of this fact. A total frequency band is specified (given). The relevant target gas frequency band of a target gas to be detected lies within this overall frequency band. The radiation source is configured as follows: The electromagnetic radiation emitted by the radiation source covers the entire frequency band. This means: At any frequency at which at least one target gas to be detected attenuates the radiation more than the lower attenuation limit, the radiation source emits radiation with an intensity above a lower intensity limit. It is possible, but not necessary, that the radiation has an intensity greater than the lower intensity limit at each frequency within the total frequency band.

According to the present disclosure, the detection arrangement is configured such that a target gas in the gas sample causes the following: At least one measuring detector generates a signal that deviates from the reference signal of this measuring detector. This applies to every target gas and to every possible concentration of this target gas being sufficiently high enough. For the embodiment just described with the photoelectric or photoacoustic measuring detectors, this means the following if at least one target gas is present in the gas sample in the measuring chamber: At least one measuring detector reacts differently to the incident radiation after the radiation has at least once penetrated the gas sample, compared with a gas sample free of each target gas. For example, a respective wavelength filter is present in front of at least one measuring detector, preferably in front of each measuring detector, and additionally in front of the optional reference detector, wherein each wavelength filter only allows radiation in a specific frequency range to pass through (transmit). It is also possible that a prism or a mirror divides radiation between at least two measuring detectors and in doing so breaks it down (splits it up) into different frequency bands.

Preferably, all M measuring detectors and additionally the optional reference detector are constructed and implemented in the same way, and the at least two different signals (first signal, deviating signal) of a measuring detector are generated as just described by way of example. In one embodiment, different wavelength filters are arranged in front of the measuring detectors. This embodiment makes it easier to manufacture the measuring detectors. They can all be constructed in the same way.

In one embodiment, the radiation source comprises at least two individual light sources, for example individual LEDs. Preferably, the number of individual light sources of the radiation source corresponds to the number M of measuring detectors of the detection arrangement or is greater than M. Each of the M inputs of the determiner is assigned to at least one individual light source of the radiation source.

Each individual light source is capable of emitting electromagnetic radiation in one light source frequency band. In this light source frequency band, the intensity of the radiation emitted by this light source is above the lower intensity limit. The light source frequency bands together (in their entirety) cover the total frequency band in the sense described above. This means: At any frequency at which at least one target gas to be detected attenuates the radiation more than the attenuation limit, the individual light sources together emit radiation with an intensity above a lower intensity limit. In many applications, this embodiment eliminates the need to emit radiation with an intensity greater than the lower intensity limit at every frequency of the overall frequency band. Therefore, this embodiment often saves electrical energy.

In an implementation of the embodiment in which the radiation source comprises at least two individual light sources, preferably M individual light sources, the gas detection device is configured as follows: At any given time point, at most one individual light source of the radiation source is switched on and emits electromagnetic radiation in a light source frequency band. The switched-on individual light source emits the electromagnetic radiation continuously or in pulsed form. The or any other individual light source of the radiation source is switched off at this time point. Preferably, each individual light source of the radiation source is temporarily switched on during use of the gas detection device.

In many cases, this implementation eliminates the need to provide a wavelength filter between the radiation source and the measuring detector. In addition, this implementation eliminates the need to provide a plurality of individual measuring detectors spaced apart from each other. Rather, it is sufficient that the detection arrangement comprises a single measuring detector. According to this implementation, at a time point at which this measuring detector supplies a signal value, only one individual light source is switched on. The signal value therefore depends on how strongly gas sample in the measuring chamber absorbs (attenuates) the electromagnetic radiation of this one switched-on individual light source, i.e., radiation in the relevant light source frequency band.

Preferably, in the embodiment just described, each individual light source is associated with one of the M inputs of the determiner, and two different individual light sources are associated with two different inputs. According to the present disclosure, the detection arrangement comprises M measuring detectors. The following notation is used for the implementation just described, in which at most one individual light source is switched on at any time point and only one measuring detector is used: At any given time point at which a particular individual light source is turned on, that measuring detector acts as a measuring detector associated with a particular one of the M inputs of the determiner. The signal that the measuring detector provides at this time point acts as the signal of the measuring detector associated with this input.

The present disclosure further relates to an arrangement with a gas detection device according to the present disclosure and a signal-processing generating device, wherein the generating device is configured to automatically generate the determiner of the gas detection device according to the present disclosure, as well as a method for generating the determiner.

The arrangement further comprises a training gas sample set. The training gas sample set comprises a plurality of gas samples. The respective composition of each gas sample in the training gas sample set is known. This means: The respective concentration of each of the N target gases to be detected in the gas sample is known. For each target gas to be detected, the training gas sample set comprises at least one gas sample containing this target gas at a concentration above a specified lower concentration limit. In a preferred embodiment, each target gas to be detected occurs in at least 50, particularly preferably in at least 100 different gas samples of the training gas sample set, these gas samples having the target gas with a concentration above the lower concentration limit. It is possible that at least one gas sample, preferably each gas sample, of the training gas sample set contains only exactly one target gas having a concentration above the lower concentration limit, i.e., is free of the or any other target gas. Optionally, the training gas sample set additionally comprises at least one gas sample that is free of each target gas to be detected.

The generating device is configured to perform the following steps for each gas sample of the training gas sample set, and the method for generating the determiner comprises the following steps for each gas sample of the training gas sample set:
The gas sample is fed into the measuring chamber of the gas detection device. Preferably, the measuring chamber was rinsed out beforehand.
Each measuring detector generates a first signal. This first signal correlates with the chemical composition of the gas sample, i.e. the N concentrations of the N target gases to be detected. Therefore, there are M signals of the measuring detectors for the gas sample.
The optional reference detector also generates a signal.
A training sample element is generated. This training sample element comprises, on the one hand, for each target gas to be detected, a respective identification of the concentration of this target gas in the gas sample, i.e. N identifications for target gas concentrations. As mentioned above, the chemical composition of the gas sample is known. On the other hand, this training sample element comprises, for each measuring detector, a respective identification of the first signal that this measuring detector has generated for this gas sample, i.e. M identifications of first signals.

Furthermore, the generating device is configured to carry out the following step and the method comprises the following step:

The determiner is trained automatically by applying a machine learning method to a training sample. The training sample used for training comprises the training sample elements generated as just described, namely using the gas samples of the training gas sample set.

The aforementioned advantages for such a gas detection device also apply to this arrangement.

In one embodiment, the generating device is configured to generate a trained matrix A wherein the trained matrix A comprises M rows for the M measuring detectors and N columns for the N target gases. To generate the trained matrix A, the generating device generates for every gas sample of the training gas sample set the respective sample element a vector sample element. Each vector sample element comprises a sample concentration vector x with N vector elements and a sample measurement vector b with M vector elements. The M vector elements of the sample measurement vector b depend on the signals of the M measuring detectors provided for this gas sample, and the N vector elements of the sample concentration vector x depend on the concentrations of the N target gases in the gas sample. The generating device generates the matrix such that the product A x deviates for the vector sample elements as little as possible from the vector b. For doing so the generating device uses the vector sample elements. For generating the matrix A, the generating device preferably applies a minimization method on in error function, preferably an iterative minimization method. The error function yields for every vector sample element an indicator for the difference (deviation) between the product A x and the vector b.

### BRIEF DESCRIPTION OF THE DRAWINGS

In the following, the present disclosure is described on the basis of an exemplary embodiment. In the drawings,

FIG. 1 schematically shows a first embodiment of the gas detection device according to the present disclosure, wherein a radiation source emits broadband electromagnetic radiation and wherein the radiation impinges on three parallel measuring detectors;

FIG. 2 schematically shows a second embodiment of the gas detection device according to the present disclosure, wherein a plurality of diodes each emit narrow-band electromagnetic radiation and the radiation impinges on three parallel measuring detectors as in FIG. 1;

FIG. 3 schematically shows a third embodiment of the gas detection device according to the present disclosure, wherein, as in FIG. 2, a plurality of diodes each emit narrow-band electromagnetic radiation and the radiation impinges on a single measuring detector;

FIG. 4 shows a signal-processing determiner with a neural network;

FIG. 5 shows the training of the determiner, wherein a trained matrix is generated;

FIG. 6 shows the determiner trained according to FIG. 5 with a pseudo-inverse of the matrix.

### DETAILED DESCRIPTION

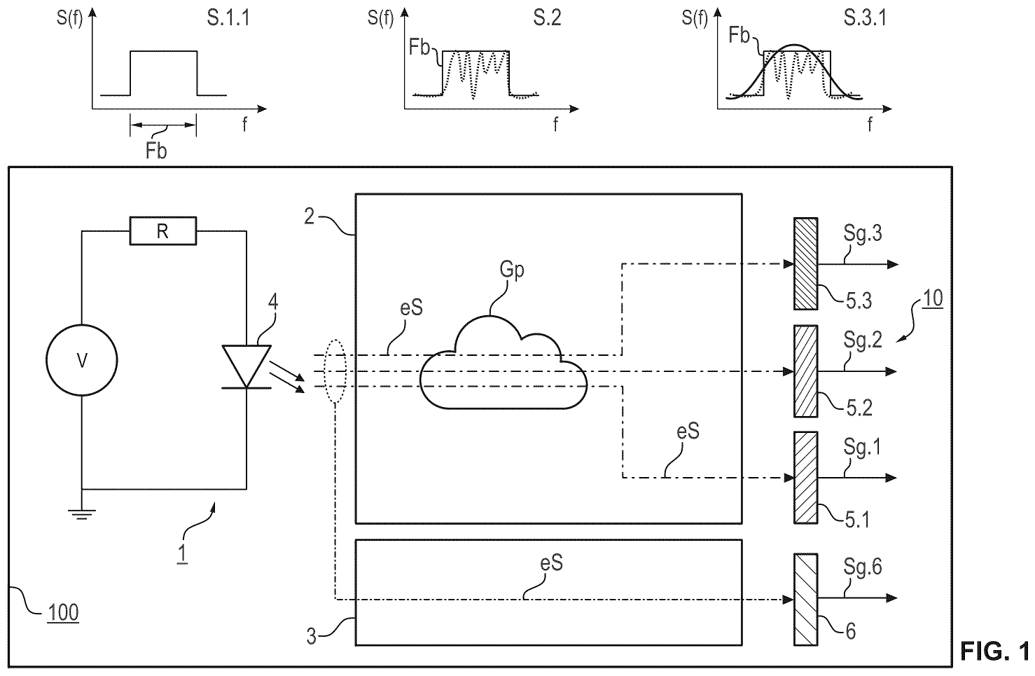

FIG. 1 to FIG. 3 schematically show three different embodiments of a gas detection device 100 according to the present disclosure. In all three embodiments, the gas detection device 100 according to the present disclosure is capable of detecting N target gases simultaneously. N is a predetermined number greater than or equal to 2, in the exemplary embodiment equal to 3.

A gas sample Gp to be examined is located in a measuring chamber 2 wherein the gas sample Gp stems from a spatial region to be monitored. Each target gas to be detected attenuates electromagnetic radiation eS in a respective target gas frequency band. The target gas frequency band may comprise at least two individual and mutually disjunctive frequency bands. An overall frequency band is specified. The relevant target gas frequency band in which a target gas to be detected is able to attenuate electromagnetic radiation eS lies within the same given overall (total) frequency band.

A radiation source 1 emits electromagnetic radiation eS. The electromagnetic radiation eS emitted by the radiation source 1 covers the overall frequency band. More specifically, this means the following for each frequency in the overall frequency band: At least if the sum of the absorption coefficients of all N target gases to be detected at this frequency is greater than a predetermined lower absorption limit, the radiation source 1 emits electromagnetic radiation eS at this frequency with a radiation intensity (strength) above a predetermined lower radiation intensity limit. This lower radiation intensity limit is chosen such that absorption above the lower absorption limit can be reliably detected.

A part of the emitted electromagnetic radiation eS enters the measuring chamber 2 and penetrates at least once the gas sample Gp in the measuring chamber 2. Optionally, the electromagnetic radiation eS is reflected at least once and penetrates the gas sample Gp in the measuring chamber 2 at least twice. After the electromagnetic radiation eS has penetrated the gas sample Gp at least once, it impinges onto (hits) three measuring detectors 5.1, 5.2, 5.3, which are arranged parallel to the radiation eS. Another part of the electromagnetic radiation eS is passed past the measuring chamber 2 and thus past the gas sample Gp and through a reference chamber 3 and impinges onto a reference detector 6.

The three measuring detectors 5.1, 5.2, 5.3 and the reference detector 6 belong to a detector arrangement 10.

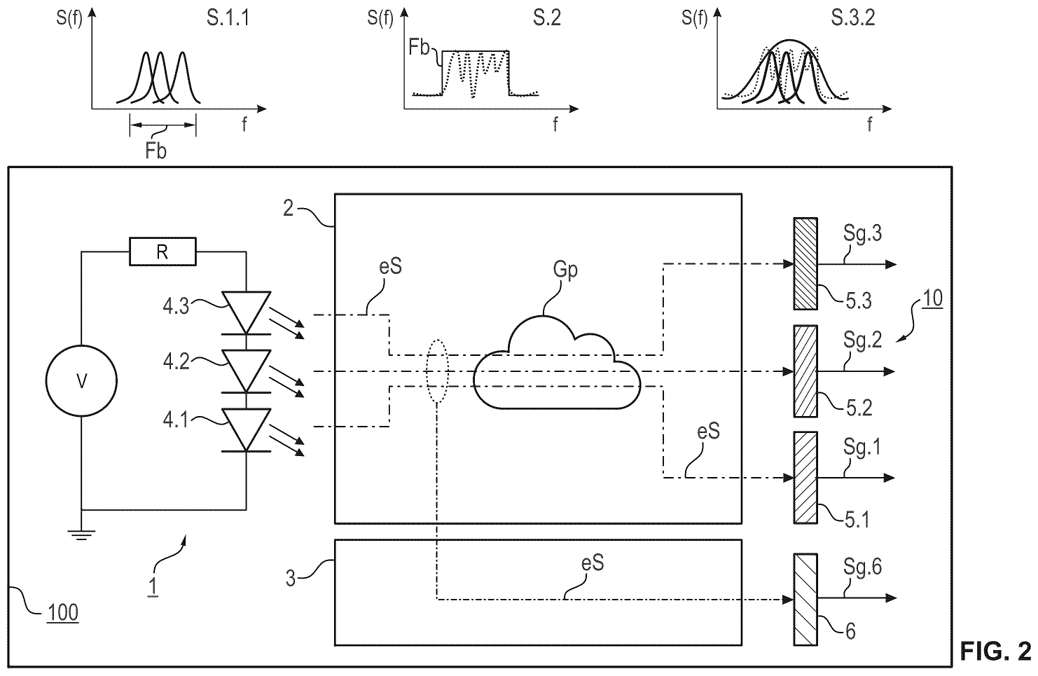

In the embodiment according to FIG. 1 and FIG. 2, each measuring detector 5.1, 5.2, 5.3 is excited by incident electromagnetic radiation eS only in a specific detector frequency band. In other words: The signal of the measuring detector 5.1, 5.2, 5.3 is only greater than a predetermined lower limit if the frequency of the incident radiation is in the relevant detector frequency band. This property is achieved, for example, by an appropriate wavelength filter (not shown) upstream of the relevant measuring detector 5.1, 5.2, 5.3. The detector frequency bands also cover the overall frequency band. Apart from the wavelength filter, the three measuring detectors 5.1, 5.2, 5.3 can be constructed in the same way.

The three measuring detectors 5.1, 5.2, 5.3 each generate a respective first signal Sg.1, Sg.2, Sg.3; the reference detector 6 a reference signal Sg.6. Each signal Sg.1, Sg.2, Sg.3, Sg.6 correlates with the intensity of incident (impinging) electromagnetic radiation eS.

In the first embodiment according to FIG. 1, a radiation source 4, for example an IR diode, emits broadband electromagnetic radiation eS within a frequency band Fb. The diagram S.1.1 of FIG. 1 shows schematically and in an idealized manner the frequency spectrum S(f) of the broadband radiation es, which covers the overall frequency band.

In the second embodiment according to FIG. 2, three diodes 4.1, 4.2, 4.3 arranged in parallel each emit respective electromagnetic radiation es within a respective frequency spectrum S(f). Diagram S.1.2 of FIG. 2 shows the overlay (superposition) of these three frequency spectra. The overlay also covers the overall frequency band. An advantage of the second embodiment is the following one: In many cases, the second embodiment requires less electrical energy than the first embodiment. One reason for this is the following: The emitted electromagnetic radiation eS does not necessarily have to cover the overall frequency band, but only those ranges of the entire frequency band in which at least one target gas to be detected absorbs radiation sufficiently strongly. In many cases, this requirement can be met by the second embodiment using less electrical energy than the first embodiment. This advantage is particularly important if the gas detection device 100 cannot be permanently connected to a stationary power supply network, but instead comprises its own power supply unit.

Of course, radiation sources other than infrared light sources are also possible, for example ultraviolet light sources or ultrasound sources.

Diagram S.2 shows, by way of example, the intensity of the electromagnetic radiation eS if broadband radiation eS is emitted as illustrated in diagram S.1.1 and after the electromagnetic radiation eS has penetrated the gas sample Gp. The diagrams S.3.1 and S.3.2 illustrate the intensity of electromagnetic radiation eS incident (impinging) on the measuring detectors 5.1, 5.2, 5.3. The respective output signal Sg.1, Sg.2, Sg.3 of a measuring detector 5.1, 5.2, 5.3 depends on the respective concentration of each target gas. As already mentioned, a wavelength filter ensures that a measuring detector 5.1, 5.2, 5.3 is only slightly or even not at all excited by incident electromagnetic radiation eS outside a detector frequency band.

The reference detector 6 also generates a signal Sg.6, which depends on the intensity of incident electromagnetic radiation eS. Ideally, the signal Sg.6 of the reference detector 6 does not depend at all on the concentration of a target gas in the spatial region to be monitored. Ideally, the reference signal Sg.6 of the reference detector 6 is influenced in the same way as the respective first signal of a measuring detector 5.1, 5.2, 5.3 by environmental influences, such as ambient pressure, ambient temperature, and ambient humidity, as well as by the current state of the gas detection device 100, for example the state of the radiation source 1 and of an optional power supply unit. Therefore, using the signal from the reference detector 6, the effects of environmental influences and of the state of the gas detection device 100 on the respective signal of a measuring detector 5.1, 5.2, 5.3 are computationally compensated for. For example, a quotient Sg.1/Sg.6, Sg.2/Sg.6, Sg.3/Sg.6 is calculated and used. This quotient is referred to as the normalized signal of the relevant measuring detector 5.1, 5.2, 5.3.

In the two embodiments according to FIG. 1 and FIG. 2, the detector arrangement 10 comprises three measuring detectors 5.1, 5.2, 5.3 arranged in parallel. Each measuring detector 5.1, 5.2, 5.3 is excited by incident (impinging) electromagnetic radiation eS only within a specific detector frequency band. In the first embodiment according to FIG. 1, a radiation source 4 emits broadband electromagnetic radiation eS. In the second embodiment according to FIG. 2, three parallel radiation sources 4.1, 4.2, 4.3 emit narrowband electromagnetic radiation eS in three different frequency spectra. The three radiation sources 4.1, 4.2, 4.3 can emit electromagnetic radiation eS continuously or in a pulsed and temporally overlapping manner.

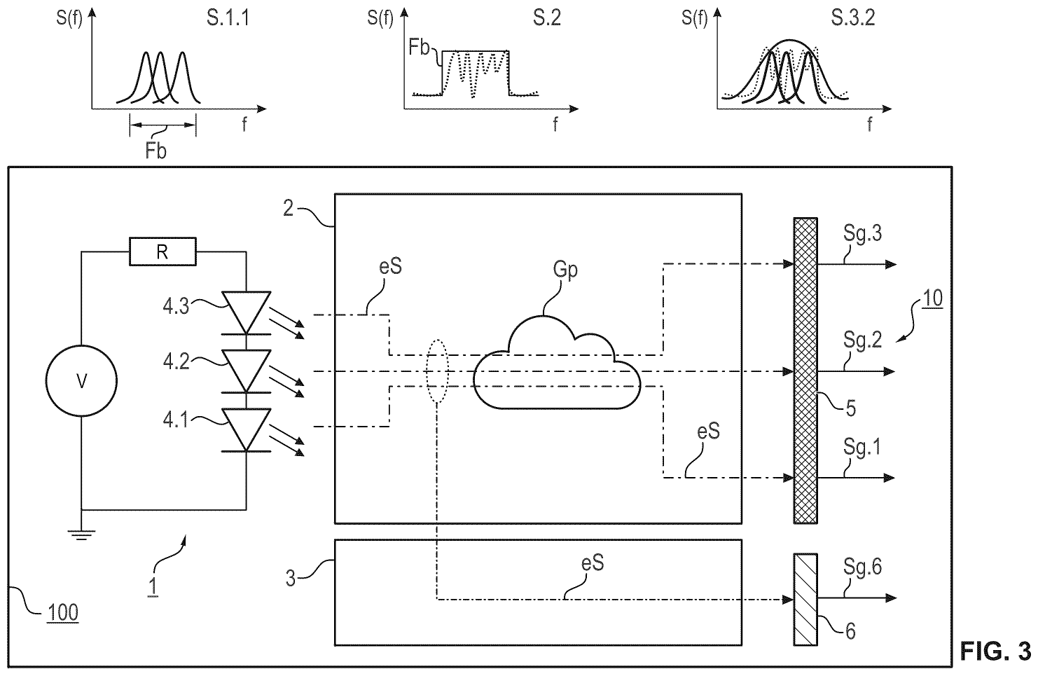

FIG. 3 shows an alternative third embodiment. Just as in the embodiment according to FIG. 2, the gas detection device 100 in the third embodiment also comprises three parallel radiation sources 4.1, 4.2, 4.3, which emit electromagnetic radiation eS in three different frequency spectra. Instead of three parallel measuring detectors 5.1, 5.2, 5.3, by contrast the detector arrangement 10 comprises a single measuring detector 5 as well as a reference detector 6. This single measuring detector 5 also generates an output signal depending on the intensity of incident electromagnetic radiation eS. A wavelength filter is not necessarily arranged between the radiation sources 4.1, 4.2, 4.3 and the measuring detector 5.

According to the third embodiment, the three radiation sources 4.1, 4.2, 4.3 can be switched on and off independently of each other. A switched-on radiation source 4.1, 4.2, 4.3 emits continuous or pulsed electromagnetic radiation. At any time point, no more than one radiation source 4.1 or 4.2 or 4.3 is switched on and the other radiation sources are switched off. While the gas detection device 100 monitors a spatial region, each one of the three radiation sources 4.1, 4.2, 4.3 is temporarily switched on.

In order to use a uniform notation for all three embodiments, the following notation is used for the third embodiment: The measuring detector 5 is designated 5.x while the radiation source 4.x is switched on and every other radiation source 4.y is switched off. Sg.x denotes the signal that the measuring detector 5 supplies if and while the radiation source 4.x is switched on and every other radiation source 4.y is switched off (x=1, 2, 3, y # x).

In order to use a uniform notation for all three embodiments, the following notation is used for the third embodiment with the single measuring detector 5: The measuring detector 5 is denoted with 5.x if the radiation source 4.x is switched-on and every other radiation source 4.y is switched off. Sg.x denotes that first signal which the measuring detector 5 provides if the radiation source 4.x is switched on and every other radiation source 4.y is switched off (x=1, 2, 3, y # x).

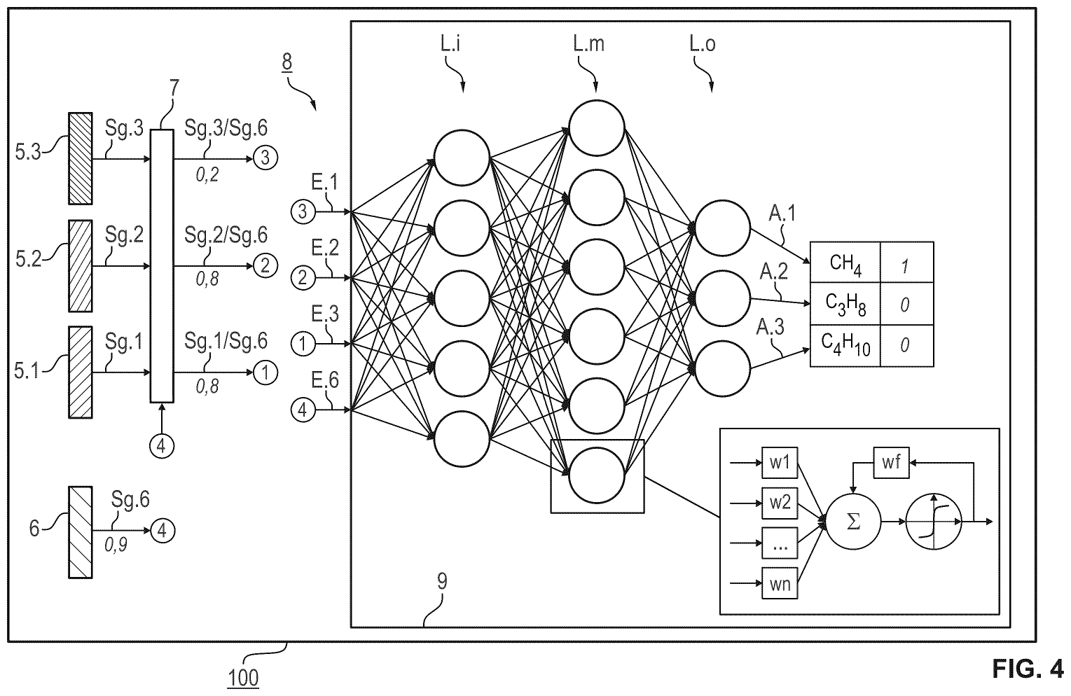

The gas detection device 100 comprises a signal-processing evaluation unit 8, see FIG. 4. This evaluation unit 8 supplies, for each target gas to be detected, at least the statement (information) as to whether this target gas occurs in the gas sample Gp with a concentration above a predetermined lower concentration limit, optionally the respective concentration with which this target gas appears in the gas sample Gp. The target gas concentration determined by the evaluation unit 8 is usually only an approximation of the actual (real) target gas concentration in the spatial region to be monitored.

A normalizer 7 of the signal processing evaluation unit 8 normalizes the three first signals from the three measuring detectors 5.1, 5.2, 5.3. For example, the normalizer 7 divides the respective first signal of a measuring detector 5.1, 5.2, 5.3 by the signal of the reference detector 6. A signal-processing determiner 9 of the evaluation unit 8 comprises one respective input for each measuring detector 5.1, 5.2, 5.3, optionally an additional input for the reference detector 6.

In both embodiments according to FIG. 4 to FIG. 6, the determiner 9 has four inputs E.1, E.2, E.3, E.6. The respective normalized signal Sg.1/Sg.6, Sg.2/Sg.6, Sg.3/Sg.6 of a measuring detector 5.1, 5.2, 5.3 is present at an assigned one of the first three inputs E.1, E.2, E.3, and the signal Sg.6 of the reference detector 6 is present at the fourth input E.6. The input E.6 for the signal Sg.6 of the reference detector 6 is an optional input.

In the example shown, the gas detection device 100 is configured to detect the three combustible target gases methane (CH₄), propane (C₃H₈), and butane (C₄H₁₀) in a gas sample Gp. In the example shown, the determiner 9 has three outputs A.1, A.2, A.3, namely one output A.1, A.2, A.3 for each target gas CH₄, C₃H₈ and C₄H₁₀ to be detected. The target gases and the number M=3 of assigned inputs and the number N=3 of outputs are only examples. Of course, other target gases and a different number of outputs are also possible. The determiner 9 provides the determined concentration of the associated target gas at a respective assigned output A.1, A.2, A.3.

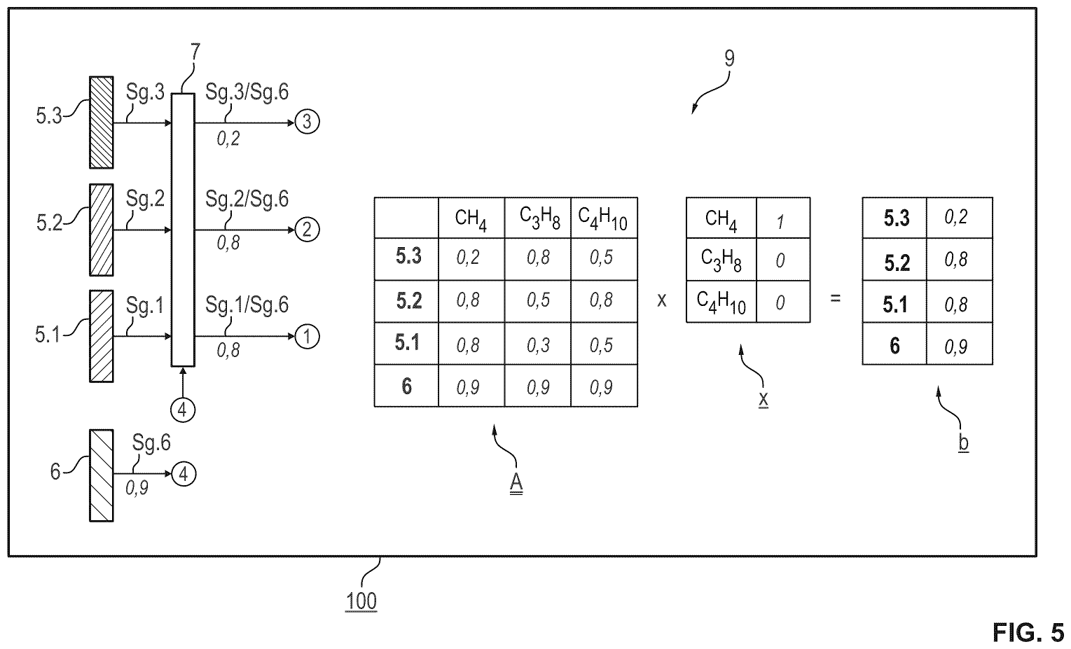

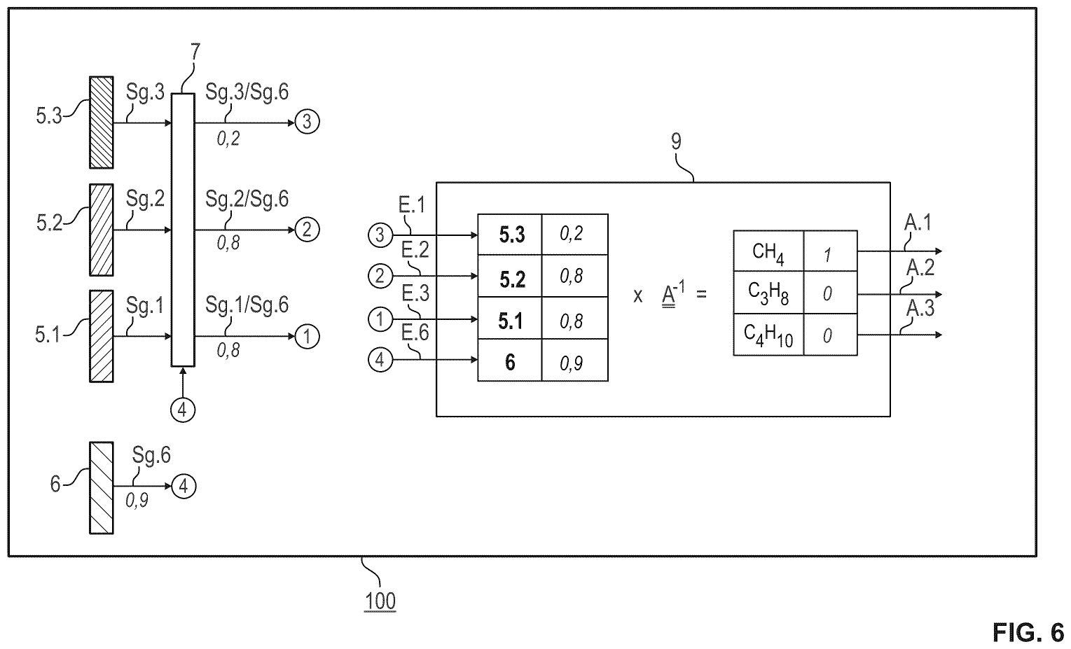

Both in the embodiment according to FIG. 4 and in the embodiment according to FIGS. 5 and 6, the determiner 9 is trained in advance with the help of a computer-evaluable training sample. This training sample comprises a plurality of sample elements. Each sample element was generated using a gas sample Gp. The gas sample Gp used to generate a sample element has a known chemical composition. This means: The respective concentration of each target gas to be detected in the gas sample Gp is known. Each gas sample Gp is fed or otherwise conveyed into the measuring chamber 2, the radiation source 1 emits electromagnetic radiation eS, and the resulting signal Sg.1, Sg.2, Sg.3, Sg.6 of each detector 5.1, 5.2, 5.3, 6 is recorded and stored. Preferably, for each target gas to be detected, at least K gas samples are generated and measured, where K is greater than or equal to 100 and these K gas samples comprise only this one target gas and are free of the target gas or any other target gas to be detected. However, it is also possible that the gas sample of at least one sample element contains two different target gases having a sufficiently high target gas concentration.

N denotes the number of target gases to be detected and M the number of measuring detectors. In this case M=N=3 holds. Each sample element comprises N values for the N target gas concentrations in the gas sample Gp having known chemical composition that led to this sample element. Furthermore, each sample element comprises M values for the M signals Sg.1, Sg.2, Sg.3 of the M measuring detectors yielded when analyzing this gas sample. By way of example, FIGS. 4 to 6 show the three normalized signal values Sg.1=0.8, Sg.2=0.8, Sg.3=0.2 of the three measuring detectors 5.1, 5.2, 5.3 as well as the signal value Sg.6=0.9 of the reference detector 6.

In the example shown in FIG. 4 to FIG. 6, each gas sample Gp used to generate the training sample has only one target gas above a lower concentration limit. In the example shown, on the one hand each signal Sg.1, Sg.2, Sg.3 of a measuring detector 5.1, 5.2, 5.3 is normalized with the signal of the reference detector 6. On the other hand, the normalized signal Sg.1/Sg.6, Sg.2/Sg.6, Sg.3/Sg.6 is divided by the relevant target gas concentration. The N values for the target gas concentrations are therefore equal to 1 once and equal to 0 (N−1) times. By way of example, FIG. 4 and FIG. 5 show the value 1 for methane and the value 0 for butane and propane.

In the example of FIG. 4, the classifier 9 comprises an artificial neural network (ANN). This neural network is trained using the training sample. The neural network has M inputs E.1, E.2, E.3 for the M measuring detectors 5.1, . . . , optionally another input E.6 for the reference detector 6, and N outputs A. 1, A.2, A.3 for the N target gases to be detected. The neural network 9 comprises an input layer L.i, an intermediate layer L.m, and an output layer L.o. The neural network can be trained using the back propagation algorithm. Using back propagation is preferred. FIG. 4, bottom right, shows an example of a node (a functional unit, a “perceptron”) of the neural network. The node has n inputs and forms a weighted mean of the n values at its n inputs using n weighting factors w1, . . . , wn. The node applies a nonlinear function, shown by way of example, to this weighted mean. This function is, for example, a threshold function or more generally an activation function. Optionally, the result is fed back once, with a weighting factor wf.

Preferably, the training sample is divided into two sets of sample elements. The first set is used to train the neural network 9. The second set is used to check the trained neural network 9. If the deviation between the results of the neural network 9 and the actual target gas concentrations is too large, retraining is performed, preferably with another (a further) training sample.

An advantage of the embodiment implementing the classifier 9 using a neural network is the following: It is not necessary to make an assumption about the influence of different target gases on the gas sample Gp. Furthermore, it is not necessary to make an assumption about the cross-sensitivity or lack of cross-sensitivity of different target gases on the signals of the measuring detectors 5.1, 5.2, 5.3. One disadvantage is the following: The neural network 9 is effectively a black box. The weighting factors and activation functions usually have no physical meaning. It is not possible to explain or analytically verify a result of the neural network 9.

In the embodiment according to FIG. 5 and FIG. 6, the following model assumptions are made: The target gases in a gas sample Gp attenuate electromagnetic radiation eS of a specific frequency in the measuring chamber 9 independently from each other. The influences of two different target gases on electromagnetic radiation eS of a frequency are additively superimposed. Cross-sensitivities between the target gases do not occur to any relevant extent. In many cases, these model assumptions apply with sufficient accuracy.

FIG. 5 illustrates an example of how the determiner 9 is trained, and FIG. 6 illustrates an example of the application of the trained determiner 9. During training according to FIG. 5, a trained matrix A is generated by applying the training sample. The matrix A comprises one respective row (line) for each measuring detector 5.1, 5.2, 5.3, and for each target gas CH₄, C₃H₈, C₄H₁₀ to be detected one respective column. Optionally, the trained matrix A comprises an additional row for the reference detector 6. In the example shown, the matrix A has M+1=4 rows and N=3 columns.

The sample elements of this embodiment are denoted by vector sample elements. Each sample element consists of a sample concentration vector x having N vector elements, namely one vector element per target gas to be detected, and a sample measurement vector b having M+1 vector elements for the M measuring detectors 5.1, 5.2, 5.3 and for the reference detector 6. In the example shown, the signal values for a gas sample Gp are entered into the trained matrix A, which values, as just described, are normalized with the signal value Sg.6 of the reference detector 6 and with the target gas concentration of the only target gas in the gas sample (in this case: methane). The sample concentration vector x consists of a 1 for methane and a 0 in each case for propane and butane. The sample measurement vector b consists of the three standardized signal values Sg.1/Sg.6, Sg.2/Sg.6, Sg.3/Sg.6 of the three measuring detectors 5.1, 5.2, 5.3 and the signal value Sg.6 of the reference detector 6.

In an alternative embodiment, at least one zero point measurement and one zero point calibration are carried out beforehand (in advance). During the zero point measurement or during each zero point measurement, a gas sample containing no target gas is fed to the measuring chamber 9. Also in this situation, each measuring detector 5.1, 5.2, 5.3 supplies a signal value which is used as zero value. These zero values are denoted by Sg.1_{Null}, Sg.2_{Null}, Sg.3_{Null}, Sg.6_{Null}. The measurements just described with different gas samples, each containing a single target gas, supply raw signal values Sg.1_{Roh}, Sg.2_{Roh}, Sg.3_{Roh}, Sg.6_{Roh}. The differences Sg.i=Sg.i_{Roh}−Sg.i_{Null} (i=1,2,3,6) are used as signal values for the procedure according to FIG. 4 to FIG. 6.

FIG. 5 shows an ideal situation wherein each of the K gas samples containing only methane leads to the same sample measurement vector b. In practice, these K gas samples lead to up to K different sample measurement vectors. The same applies to any other target gas to be detected. Therefore, for each target gas i (i=1, . . . , N) an averaged sample measurement vector bₘₑₐₙ(i) is calculated. To calculate this sample measurement vector bₘₑₐₙ(i), b is averaged using the K vectors, which are generated as a reaction on measuring the K gas samples with the target gas i. The averaged sample measurement vector bₘₑₐₙ(i) is used as column i of matrix A, where column i stands for the target gas i (i=1, . . . , N).

While using the gas detection device 100, a signal value vector b is generated from the signals of the M+1 detectors 5.1, 5.2, 5.3, 6, see FIG. 6. The unknown and desired chemical composition of the gas sample Gp in the measuring chamber 2 is determined by the equation x=A⁻¹ b. As a rule, the trained matrix A is not symmetrical. The matrix A⁻¹ is the inverse or pseudo-inverse of the trained matrix A. Various methods are possible to calculate the pseudo-inverse of the trained matrix A, in particular
singular value decomposition (SVD),
Thikonov regularization,
Kaczmarz algorithm.

In one embodiment, the pseudo-inverse A⁻¹ is generated and saved once in advance by inverting the trained matrix A and is used as part of the determiner 9. In another embodiment, the pseudo-inverse A⁻¹ is calculated during runtime, preferably iteratively and until a predetermined termination criterion is met.

List of reference signs

| | |
|---|---|
| 1 | Radiation source, emits electromagnetic radiation eS, |
| | comprises in one embodiment the radiation source 4 and in |
| | another embodiment the diodes (individual light sources) |
| | 4.1, 4.2, 4.3 |
| 2 | Measuring chamber, contains a gas sample Gp to be |
| | examined, is penetrated by electromagnetic radiation eS |
| 3 | Reference chamber, is penetrated by electromagnetic |
| | radiation eS |
| 4 | Broadband emitting IR radiation source |
| 4.1, 4.2, | Narrow-band emitting IR LEDs, act as individual light |
| 4.3 | sources |
| 5.1, 5.2, | Measuring detectors, each generate a signal Sg.1, Sg.2, |
| 5.3 | Sg.3 which depends on the intensity of incident |
| | electromagnetic radiation eS |
| 6 | Reference detector, generates the signal Sg.6 |
| 7 | Normalizer, normalizes the signals of the measuring |
| | detectors 5.1, 5.2, 5.3 with the signal of the reference |
| | detector 6 |
| 8 | Signal processing evaluation unit, comprises the normalizer |
| | 7 and the determiner 9 |
| 9 | Signal processing determiner, has the M inputs E.1, E.2, |
| | E.3, the additional input E.6 and the N outputs A.1, A.2, A.3 |
| | and comprises in one embodiment the pseudo-inverse A⁻¹ |
| 10 | Detector arrangement, comprises the measuring detectors |
| | 5.1, 5.2, 5.3 and the reference detector 6 |
| A | Matrix with M + 1 rows and N columns, is generated using |
| | the training sample |
| A⁻¹ | Pseudo-inverse of the matrix A |
| A.1, A.2, | N outputs of the determiner 9, each associated with a target |
| A.3 | gas |
| E.1, E.2, | M inputs of the determiner 9, each associated with a |
| E.3 | measuring detector 5.1, 5.2, 5.3 |
| E.6 | Additional input of the determiner 9, associated with the |
| | reference detector 9 |
| eS | Electromagnetic radiation emitted by the radiation source 1, |
| | partially penetrates the gas sample Gp in the measuring |
| | chamber 2 and is partially passed through past the |
| | measuring chamber 2 and through the reference chamber 3 |
| Fb | Frequency band in which the radiation source 1 emits |
| | electromagnetic radiation eS |
| Gp | Gas sample in the measuring chamber 2 is penetrated by |
| | electromagnetic radiation eS |
| K | Number of gas samples of the training gas sample set, |
| | each have exactly one target gas to be detected, wherein |
| | each gas sample supplies one sample element |
| L.i | Input layer of neural network 9, has M + 1 inputs |
| L.m | Intermediate layer of the neural network 9 |
| L.o | Output layer of neural network 9, has N outputs |
| M | Number of measuring detectors, in this case: M = 3 |
| N | Number of target gases to be detected, in this case: N = 3 |
| S.1 | Frequency spectrum of the radiation source 1 |
| S.1.1 | Frequency spectrum of the radiation source 1 for a |
| | broadband emitting radiation source 4 |
| S.1.2 | Frequency spectrum of radiation source 1 with three |
| | narrowband emitting diodes 4.1, 4.2, 4.3 |
| S.2 | Absorption spectrum of the gas sample Gp for broadband |
| | radiation |
| S.3 | Frequency spectrum of the radiation eS incident on the |
| | detectors 5.1, 5.2, 5.3 |
| S.3.1 | Frequency spectrum of the measuring detectors 5.1, 5.2, |
| | 5.3 for a broadband emitting radiation source 4 |
| S.3.2 | Frequency spectrum of the measuring detectors 5.1, 5.2, |
| | 5.3 for three narrowband emitting diodes 4.1, 4.2, 4.3 |
| Sg.1 | Signal of the measuring detector 5.1 |
| Sg.2 | Signal of the measuring detector 5.2 |
| Sg.3 | Signal of the measuring detector 5.3 |
| Sg.6 | Signal of the reference detector 6 |

1-16. (canceled)

17. A gas detection device for detecting several predetermined target gases in a gas sample, comprising:
a measuring chamber;
a detection arrangement having several measuring detectors; and
an evaluation device having a signal-processing determiner,
wherein the measuring chamber is configured to hold a gas sample to be analyzed,
wherein each measuring detector is configured to generate a respective first signal, wherein each first signal correlates to a concentration of at least one target gas of the several predetermined target gases in the gas sample,
wherein the detection arrangement is configured such that:
if the gas sample is free of any target gas of the several predetermined target gases, each measuring detector of the detection arrangement generates a respective reference signal; and
for each different chemical composition of the gas sample, at least one measuring detector of the detection arrangement generates a deviating signal that deviates from the respective reference signal of this measuring detector,
wherein the first signal of a given measuring detector is the reference signal or the deviating signal,
wherein the determiner comprises:
a plurality of inputs comprising, for each measuring detector of the detection arrangement one associated input; and
a plurality of outputs comprising, for each target gas of the several predetermined target gases, one associated output,
wherein the gas detection device is configured such that:
at each associated input of the determiner a respective third signal is applied, wherein each third signal depends on the first signal of the associated measuring detector of the detection arrangement; and
each associated output of the determiner provides information about a concentration of the respective associated target gas,
wherein the determiner is trained by applying a machine-learning method to a given training sample,
wherein the given training sample comprises a plurality of sample elements, wherein each sample element comprises:
for each target gas of the several predetermined target gases, a concentration value that depends on a concentration of said target gas and
for each measuring detector of the detection arrangement, a measurement value that depends on the respective first signal which said measuring detector generates for the combination of the concentrations of the predetermined target gases in said sample element.

18. The gas detection device of claim 17,
wherein the determiner is configured to provide at its associated outputs a concentration vector, wherein the concentration vector is an arithmetic product of an inverse or a pseudoinverse of a trained matrix with a measurement vector, every value of the concentration vector comprising information about a concentration of the respective associated target gas,
wherein every element of the measurement vector is associated with a respective measuring detector of the detection arrangement and depends on the respective first signal generated by the associated measuring detector,
wherein the trained matrix;
comprises, for every measuring detector of the detection arrangement an associated row and, for every predetermined target gas of the several predetermined target gases, an associated column; and
is generated such that for the given training sample an indicator of a deviation between:
a product of the trained matrix and the measurement vector, and
the concentration vector,
is minimized, and
wherein every sample element of the given training sample comprises a respective concentration vector and a respective measurement vector.

19. The gas detection device of claim 17,
wherein the detection arrangement comprises a reference detector,
wherein the reference detector is configured to generate a fourth signal, wherein the fourth signal:
is based on one or more of:
ambient conditions; or
a state of the gas detection device; and
is independent of a chemical composition of the gas sample in the measuring chamber.

20. The gas detection device of claim 19, wherein the gas detection device is configured such that:
a given signal, which is applied to a given input, of the determiner, wherein the given input is associated with a given measuring detector of the detection arrangement, depends on:
the respective first signal of the associated given measuring detector; and
the fourth signal,
wherein:
a value of the given signal, applied to the given input, increases proportional to the respective first signal of the associated given measuring detector and increases inversely proportional to the fourth signal, or
the value of the given signal, applied to the given input, increases inversely proportional to the respective first signal and increases proportional to the fourth signal.

21. The gas detection device of claim 19:
wherein the determiner comprises an additional input,
wherein the additional input is associated with the reference detector, and
wherein the gas detection device is configured such that the fourth signal is applied to the additional input.

22. The gas detection device of claim 17,
wherein each target gas of the several predetermined target gases attenuates electromagnetic radiation in a respective target gas frequency band,
wherein a total frequency band comprising each target gas frequency band is given,
wherein the gas detection device comprises a radiation source,
wherein the radiation source is configured to emit electromagnetic radiation,
wherein a frequency band of the emitted electromagnetic radiation covers the total frequency band,
wherein the gas detection device is configured such that at least a portion of the emitted electromagnetic radiation penetrates at least once the measuring chamber and, after penetrating, impinges on the detection arrangement, and
wherein each measuring detector of the detection arrangement is configured to generate, as the respective first signal, a signal that depends on an intensity of incident electromagnetic radiation.

23. The gas detection device of claim 22:
wherein the radiation source comprises at least two individual light sources,
wherein each individual light source of the radiation source is configured to emit electromagnetic radiation in a respective light source frequency band, and
wherein the respective light source frequency bands together cover the total frequency band in such a way that:
for a frequency range, in which at least one target gas of the several predetermined target gases attenuates electromagnetic radiation more than a predetermined lower attenuation limit,
the electromagnetic radiation emitted by the individual light sources has a total intensity in the frequency range that is greater than a predetermined lower intensity limit.

24. The gas detection device of claim 23:
wherein the gas detection device is configured such that at any time point at most one individual light source of the radiation source is switched on and one or more other individual light source of the radiation source are switched off.

25. The gas detection device of claim 17, further comprising:
an output unit,
wherein the output unit is configured to output the concentration of the respective associated target gas in at least one form which is perceptible by a human, the concentration being indicated in the information provided by the respective output of the determiner.

26. The gas detection device of claim 17, further comprising:
an alarm unit,
wherein for every target gas of the predetermined target gases a respective value range is specified,
wherein the gas detection device generates an alarm if a concentration of at least one target gas is outside the respective value range, the concentration being indicated in the information provided by the respective output of the determiner, and
wherein the alarm unit is configured to output the alarm in at least one form which is perceptible by a human.

27. An arrangement comprising:
a gas detection device for detecting several predetermined target gases in a gas sample;
a generating device; and
a training gas sample set with a plurality of gas samples;
wherein the gas detection device comprises a measuring chamber, a detection arrangement having several measuring detectors, and
an evaluation device having a signal-processing determiner,
wherein the measuring chamber is configured to hold a gas sample to be analyzed,
wherein each measuring detector is configured to generate a respective first signal,
wherein the determiner comprises:
a plurality of inputs comprising, for each measuring detector of the detection arrangement, one associated input; and
a plurality of outputs comprising, for each target gas of the predetermined target gases, one associated output,
wherein the gas detection device is configured such that at each associated input of the determiner a respective third signal is applied, wherein each respective third signal depends on the first signal of the associated measuring detector of the detection arrangement, and
wherein a respective chemical composition of each gas sample of the training gas sample set is known and each target gas to be detected occurs in at least one gas sample of the plurality of gas samples, and each associated output of the determiner provides information about a concentration of the respective associated target gas;
wherein the generating device is configured to carry out, for each gas sample of the training gas sample set, the steps of:
causing the gas sample to be guided into the measuring chamber of the gas detection device;
causing each measuring detector of the detection arrangement to generate the respective first signal for said gas sample; and
generating a training sample element, wherein the training sample element comprises:
for each given target gas of the predetermined target gases, an identification of the concentration of the given target gas in said gas sample; and
for each measuring detector of the detection arrangement, an identification of the respective first signal that this measuring detector has generated for said gas sample,
wherein the generating device is further configured to train the determiner by applying a machine learning method to a training sample, and
wherein the training sample comprises the generated training sample elements.

28. The arrangement of claim 27:
wherein the generating device is configured to generate, for each gas sample of the training gas sample set, as the respective training sample element, a respective vector sample element,
wherein the vector sample element for a gas sample comprises a sample measurement vector and a sample concentration vector,
wherein every element of the sample measurement vector is associated with a respective measuring detector of the detection arrangement and depends on the respective first signal generated by the associated measuring detector generated for said gas sample,
wherein every element of the sample concentration vector is associated with a respective target gas of the several predetermined target gases and depends on the concentration of the associated target gas in said gas sample,
wherein the generating device is further configured to generate a trained matrix,
wherein the trained matrix comprises, for every measuring detector of the detection arrangement, an associated row, and, for every predetermined target gas of the several predetermined target gases, an associated column;
wherein the generating device is configured to generate the trained matrix such that for the given training sample an indicator of a deviation between:
the product of the trained matrix and the measurement vector, and
the concentration vector,
is minimized;
wherein, in a first alternative, the generating device is configured to generate an inverse or a pseudo-inverse of the trained matrix,
wherein, in a second alternative, the determiner is configured to generate the inverse or the pseudo-inverse of the trained matrix, and
wherein, in both alternatives, the determiner is configured to provide, at its associated outputs, a concentration vector being an arithmetic product of the inverse of the trained matrix or of the pseudoinverse of the trained matrix with a measurement vector, every value of the measurement vector being associated with a respective measuring detector of the detection arrangement and depending on the respective first signal generated by the associated measuring detector, and every value of the concentration vector comprising information about a concentration of the respective associated target gas.

29. A generation method for generating a signal-processing determiner of a gas detection device:
wherein the gas detection device is configured to detect several predetermined target gases in a gas sample,
wherein the gas detection device comprises a measuring chamber, a detection arrangement having several measuring detectors, and an evaluation device comprising the determiner,
wherein the measuring chamber is configured to hold a gas sample to be analyzed,
wherein each measuring detector is configured to generate a respective first signal,
wherein the determiner comprises a plurality of inputs comprising for each measuring detector of the detection arrangement one associated input and a plurality of outputs comprising for each target gas of the several predetermined target gases one associated output,
wherein the gas detection device is configured such that at each associated input of the determiner a respective third signal is applied, wherein each third signal depends on the first signal of the associated measuring detector of the detection arrangement,
the generation method comprises:
providing a training gas sample set, the training gas sample set comprising a plurality of gas samples,
wherein a respective chemical composition of each gas sample of the training gas sample set is known and each of the several predetermined target gases occurs in at least one gas sample, and
the generation method further comprises, for each gas sample of the training gas sample set, performing the steps of:
conducting the gas sample into the measuring chamber of the gas detection device;
generating, by each measuring detector, a respective first signal which depends on the chemical composition of said gas sample; and
generating a training sample element, the training sample element comprising:
for each target gas, an identification of the concentration of the target gas in said gas sample; and
for each measuring detector, an identification of the first signal that the measuring detector has generated for said gas sample,
wherein the generation method comprises the further step of:
training the determiner by applying a machine learning method to the training sample,
wherein the training sample comprises the training sample elements generated for the gas samples of the training gas sample set.

30. The generation method of claim 29:
wherein each gas sample of the training gas sample set contains a single target gas, and
for each training sample, a respective identification of a concentration of a given target gas is:
a first identifier for the single target gas contained in the gas sample, and
a second identifier, different from the first identifier, for any other target gas.

31. The generation method of claim 29:
wherein the step of generating a respective training sample element for each gas sample of the training gas sample set comprises generating, as the training sample element for said gas sample, a vector sample element comprising:
a sample measurement vector and
a sample concentration vector,
wherein every element of the sample measurement vector is associated with a respective measuring detector of the detection arrangement and depends on the respective first signal generated by the associated measuring detector generated for said gas sample, and
wherein every element of the sample concentration vector is associated with a respective target gas of the several predetermined target gases and depends on the concentration of the associated target gas in said gas sample,
wherein the step of training the determiner by applying the machine learning method to the training sample comprises the step that:
a trained matrix is generated, the trained matrix comprising, for every measuring detector of the detection arrangement, an associated row, and, for every predetermined target gas of the several predetermined target gases, an associated column,
wherein the trained matrix is generated such that for the given training sample an indicator for a deviation between:
the product of the trained matrix and the measurement vector, and
the concentration vector,
is minimized,
wherein, in a first alternative, the generation method comprises the further step of generating an inverse or a pseudo-inverse of the trained matrix;
wherein, in a second alternative, the determiner is configured to generate the inverse or the pseudo-inverse of the trained matrix, and
wherein, in both alternatives, the determiner is configured to provide, at its associated outputs, a concentration vector being an arithmetic product of the inverse of the trained matrix or of the pseudoinverse of the trained matrix with a measurement vector, every value of the measurement vector being associated with a respective measuring detector of the detection arrangement and depending on the respective first signal generated by the associated measuring detector, and every value of the concentration vector comprising information about a concentration of the respective associated target gas.

32. The generation method of claim 31:
wherein each gas sample of the training gas sample set contains a single target gas, and
wherein a given column of the trained matrix, which column is assigned to a specific target gas of the predetermined target gases, is generated using first signals of the measuring detectors,
wherein the used first signals are generated for those gas samples which contain only said single target gas.

33. A gas detection method for detecting several predetermined target gases in a gas sample:
wherein the gas detection method is carried out using a gas detection device,
wherein the gas detection device comprises:
a measuring chamber;
a detection arrangement having several measuring detectors; and
an evaluation device having a signal-processing determiner,
wherein the detection arrangement is configured such that:
if the gas sample is free of any target gas to be detected, each measuring detector generates a reference signal, and
for each different chemical composition of the gas sample, at least one measuring detector generates a deviating signal that deviates from the reference signal of said measuring detector,
wherein the determiner comprises:
a plurality of inputs comprising, for each measuring detector of the detection arrangement, one associated input; and
a plurality of outputs comprising, for each target gas of the several predetermined target gases, one associated output,
wherein the gas detection method comprises the steps of:
conducting a gas sample to be examined into the measuring chamber;
generating, with each measuring detector, a respective first signal wherein the first signal of a measuring detector is the reference signal or the deviating signal;
applying a respective third signal to each of the inputs of the determiner, wherein each third signal depends on the respective first signal of the respective associated measuring detector; and
providing, by each of the outputs of the determiner, information about the concentration of the respective associated target gas,
wherein, in a training phase, a training gas sample set with a plurality of gas samples is provided,
wherein, in the training phase, a generation method is carried out,
wherein the training gas sample set with a plurality of gas samples is provided for the generation method,
wherein a respective chemical composition of each gas sample of the training gas sample set is known and each of the several predetermined target gases occurs in at least one gas sample,
wherein the generation method comprises, for each gas sample of the training gas sample set, the steps of:
guiding the gas sample into the measuring chamber of the gas detection device;
generating, by each measuring detector, a respective first signal for said gas sample; and
generating a training sample element which training sample element comprises:
for each of the predetermined target gases, an identification of the concentration of the target gas in said gas sample; and
for each measuring detector, an identification of the first signal that the measuring detector has generated for said gas sample,
wherein the generation method comprises the further step of training the determiner by applying a machine learning method to a training sample,
wherein the training sample comprises the generated training sample elements.

34. The gas detection method of claim 33:
wherein the step of generating a respective training sample element for each gas sample of the training gas sample set comprises generating, as the training sample element, a vector sample element comprising a sample measurement vector and a sample concentration vector,
wherein every element of the sample measurement vector is associated with a respective measuring detector of the detection arrangement and depends on the respective first signal generated by the associated measuring detector generated for said gas sample,
wherein every element of the sample concentration vector is associated with a respective target gas of the several predetermined target gases and depends on the concentration of the associated target gas in said gas sample,
wherein the step of training the determiner by applying the machine learning method to the training sample comprises the step that a trained matrix is generated,
wherein the trained matrix comprises, for every measuring detector of the detection arrangement, an associated row and, for every predetermined target gas of the several predetermined target gases, an associated column,
wherein the trained matrix is generated such that for a training sample an indicator for a deviation between:
the product of the trained matrix and the measurement vector, and
the concentration vector,
is minimized, the training sample comprising the generated training sample elements,
wherein, in a first alternative, the generation method comprises the further step of generating an inverse or a pseudo-inverse of the trained matrix;
wherein, in a second alternative, the generation method comprises the further step that the determiner generated the inverse of the trained matrix or the pseudo-inverse of the trained matrix, and
wherein, in both alternatives, the generation method comprises the further step that the determiner provides, at its associated outputs, a concentration vector being an arithmetic product of the inverse of the trained matrix or the pseudoinverse of the trained matrix with a measurement vector, every value of the measurement vector being associated with a respective measuring detector of the detection arrangement and depending on the respective first signal generated by the associated measuring detector, and every value of the concentration vector comprising information about a concentration of the respective associated target gas.
